SEQUENCE LISTING (2 sequences)

SEQ ID NO: 1 - DNA, 2391 bp, synthetic construct
  source 1..2391: mol_type = other DNA; note = CCR4-IL2 bispecific immunotoxin DNA sequence
gctggtgctgacgacgtcgtcgactcctccaagtccttcgtcatggagaacttcgcttcctaccacgggaccaagccaggttacgtcgactccatccagaagggtatccagaagccaaagtccggcacccaaggtaactacgacgacgactggaaggggttctactccaccgacaacaagtacgacgctgcgggatactctgtagataatgaaaacccgctctctggaaaagctggaggcgtggtcaaggtcacctacccaggtctgactaaggtcttggctttgaaggtcgacaacgctgagaccatcaagaaggagttgggtttgtccttgactgagccattgatggagcaagtcggtaccgaagagttcatcaagagattcggtgacggtgcttccagagtcgtcttgtccttgccattcgctgagggttcttctagcgttgaatatattaataactgggaacaggctaaggctttgtctgttgaattggagattaacttcgaaaccagaggtaagagaggtcaagatgcgatgtatgagtatatggctcaagcctgtgctggtaacagagtcagacgttctgttggttcctctttgtcctgtatcaacctagactgggacgtcatcagagacaagactaagaccaagatcgagtctttgaaagagcatggcccaatcaagaacaagatgtccgaatcccccgctaagaccgtctccgaggaaaaggccaagcaatacctagaagagttccaccaaaccgccttggagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatccatcgctttgtcctctttgatggttgctcaagctatcccattggtcggtgagttggttgacatcggtttcgctgcctacaacttcgtcgagtccatcatcaacttgttccaagtcgtccacaactcctacaaccgtccggcttactccccaggtcacaagacccaaccattcttgccatggggtggtggtggttctgacattgagttgactcaatctccatcttccttggctgtttctgctggtgagaaggttactatgtcttgtaagtcttcccaatctattttgtactcttccaaccaaaagaactacttggcttggtaccaacaaaagccaggtcaatctccaaagttgttgatttactgggcttctactagagagtctggtgttccagacagattcactggttctggttctggtactgacttcactttgactatttcttccgttcaagctgaggacttggctgtttactactgtcaccaatacttgtcttcctacactttcggtggtggtactaagttggagattaagggtggtggtggttctggtggtggtggatctggtggtggtggttctcaagttcaattgcaacaatctggtccagagttggttagaccaggtgcttctgttagaatttcttgtaaggcttctggttacactttcgcttcttactacattcaatggatgaagcaaagaccaggtcaaggtttggagtggattggttggattaacccaggtaacgttaacactaagtacaacgagaagttcaagggtaaggctactttgactgctgacaagtcttccactaccgcttacatgcaattgtcttccttgacttctgaggactctgctgtttacttctgtgctagatccacttactacagaccattggactactggggtcaaggtactaccgttactgtttcttccggtggtggtggttctggtggtggtggatccggtggtggtggttctgctccaacttcttcttctactaagaagactcaattgcaattggagcacttgttgttggacttgcaaatgattttgaacggtattaacaactacaagaacccaaagttgactagaatgttgactttcaagttctacatgccaaagaaggctactgagttgaagcacttgcaatgtttggaggaggaattgaagccattggaggaagttttgaacttggctcaatctaagaacttccacttgagaccaagagacttgatttctaacattaacgttattgttttggagttgaagggttctgagactactttcatgtgtgagtacgctgacgagactgctactattgttgagttcttgaacagatggattactttctgtcaatctattatctctactttgactcaccaccaccaccaccac

SEQ ID NO: 2 - protein, 797 aa, synthetic construct
  source 1..797: note = CCR4-IL2 bispecific immunotoxin amino acid sequence
AGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWGGGGSDIELTQSPSSLAVSAGEKVTMSCKSSQSILYSSNQKNYLAWYQQKPGQSPKLLIYWASTRESGVPDRFTGSGSGTDFTLTISSVQAEDLAVYYCHQYLSSYTFGGGTKLEIKGGGGSGGGGSGGGGSQVQLQQSGPELVRPGASVRISCKASGYTFASYYIQWMKQRPGQGLEWIGWINPGNVNTKYNEKFKGKATLTADKSSTTAYMQLSSLTSEDSAVYFCARSTYYRPLDYWGQGTTVTVSSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

(19) United States
(12) Patent Application Publication
(10) Pub. No.: US 2026/0098097 A1
(43) Pub. Date: Apr. 9, 2026

(54) IMMUNOTOXIN-BASED TARGETED THERAPY FOR CANCER

(71) Applicant: The Regents of the University of Colorado, a Body Corporate, Denver, CO (US)

(72) Inventors: Zhirui Wang, Denver, CO (US); Zhaohui Wang, Denver, CO (US); Bradley Haverkos, Denver, CO (US)

(21) Appl. No.: 19/140,326

(22) PCT Filed: Dec. 29, 2023
(86) PCT No.: PCT/US2023/086529

§ 371 Date: Jun. 17, 2025

Related U.S. Application Data
(60) Provisional application No. 63/478,045, filed on Dec. 30, 2022.

(51) Int. Cl. C07K 16/28; A61K 39/00; A61K 39/395; A61K 47/68; A61P 35/00; C07K 14/55

(52) U.S. Cl. CPC C07K 16/2866; A61K 39/3955; A61K 47/68031; A61K 47/6849; A61P 35/00; C07K 14/55; A61K 2039/505; C07K 2317/622; C07K 2317/73; C07K 2317/92; C07K 2319/55

(57) ABSTRACT

Methods of treating various types of cancer, including cutaneous T-cell lymphoma, involve administering a therapeutically effective amount of a pharmaceutical composition containing a genetically engineered C-C motif chemokine receptor 4 bispecific immunotoxin, alone, or in combination with one or more additional therapeutic agents, such as a pharmaceutical composition containing an antibody-drug conjugate.

### CROSS-REFERENCE TO RELATED APPLICATIONS

This application claims priority to U.S. Provisional Patent Application No. 63/478,045, titled “CCR4-IL2 BISPECIFIC IMMUNOTOXIN FOR TREATING CUTANEOUS T-CELL LYMPHOMA” and filed on Dec. 30, 2022, the entire contents of which are incorporated herein.

### GOVERNMENT FUNDING

This invention was made, in part, with government support under grant numbers: Colorado OEDIT (DO 2020-2666 to ZW), NHLBI/NIH (U01HL152405 to ZW), NCI/NIH (1R42CA261547-01 to ZW), and Colorado OEDIT AIA grants (CTGG1 2022-2206 to ZW). The government has certain rights in the invention.

### TECHNICAL FIELD

The present disclosure relates generally to compositions, systems, and methods for treating cancer. Specific implementations involve the delivery of a pharmaceutical composition containing an engineered immunotoxin to a subject afflicted with cancer.

### SEQUENCE LISTING

The present disclosure further incorporates by reference the Sequence Listing submitted herewith. The Sequence Listing .xml file, identified as P307749_WO_01_SL, is 6,206 bytes in size and was created on Dec. 22, 2023. The Sequence Listing, electronically filed herewith, does not extend beyond the scope of the specification, and does not contain new matter.

### BACKGROUND

Cancer affects millions of people each year, causing severe symptoms and often death. Despite substantial efforts to combat all forms of the disease, effective treatments remain elusive. The diversity of factors causing, contributing, and exacerbating various cancer types makes them extremely difficult to treat. Cutaneous T-cell lymphoma, for instance, is a heterogeneous subset of extranodal non-Hodgkin's lymphoma that includes two main subtypes: mycosis fungoides and Sezary syndrome, characterized by skin lesions resulting from the infiltration of malignant T lymphocytes. Objective response rates for the systemic treatment of mycosis fungoides and Sezary syndrome are only about 30%, and none of the existing treatments are thought to be curative. Accordingly, new cancer treatments are needed.

### SUMMARY

This disclosure provides novel methods, systems, and compositions for treating cancer. Embodiments involve administering a therapeutically effective amount of at least one pharmaceutical composition to a subject afflicted with cancer, such as cutaneous T-cell lymphoma (“CTCL”). Examples of the pharmaceutical composition may include a genetically engineered C-C motif chemokine receptor 4 (“CCR4”) bispecific immunotoxin (“CCR4-IL2 IT”). In some examples, CCR4-IL2 IT may target CCR4⁺ and CD25⁺ CTCL.

In some examples, methods may further involve administering a pharmaceutical composition containing an antibody-drug conjugate, such as an anti-CD30 antibody-drug conjugate (e.g., Brentuximab vedotin, hereinafter referred to as “anti-CD30 antibody-drug conjugate” or “antibody-drug conjugate”) to the subject afflicted with cancer. In some examples, the pharmaceutical composition containing the anti-CD30 antibody-drug conjugate may be administered concurrently with the pharmaceutical composition containing CCR4-IL2 IT. In some examples, the pharmaceutical composition containing the anti-CD30 antibody-drug conjugate and the pharmaceutical composition containing CCR4-IL2 IT may be administered separately. In some examples, the pharmaceutical composition containing the CCR4-IL2 IT may also contain the anti-CD30 antibody-drug conjugate.

In some examples, the pharmaceutical composition containing CCR4-IL2 IT reduces the number of tumor-infiltrating Treg cells in the subject. In some examples, the pharmaceutical composition is administered intravenously. In some examples, the method further involves performing a biopsy on a tumor within the subject and determining that the tumor is CD30⁺CCR4⁺ and/or CD25⁺. In some examples, the method may not involve determining whether a tumor is CD⁻, CD30⁺CCR4⁺ and/or CD25⁺. In some examples, administering to the subject the therapeutically effective amount of the pharmaceutical composition causes a reduction in skin rashes, skin lesions, tumor volume, tumor weight, tumor number, tumor metastasis, or combinations thereof.

In accordance with embodiments of the present disclosure, a pharmaceutical composition formulated for treating cutaneous T-cell lymphoma in a subject includes CCR4-IL2 IT and an anti-CD30 antibody-drug conjugate, along with one or more excipients.

In accordance with embodiments of the present disclosure, methods of treating or alleviating at least one symptom of cutaneous T-cell lymphoma in a subject may involve administering to the subject a therapeutically effective amount of a genetically engineered C-C motif chemokine receptor 4 bispecific immunotoxin (“CCR4-IL2 bispecific immunotoxin” or “CCR4-IL2 IT”) and a therapeutically effective amount of an anti-CD30 antibody-drug conjugate. In some examples, the therapeutically effective amount of the CCR4-IL2 bispecific immunotoxin is administered via a first pharmaceutical composition. In some examples, the therapeutically effective amount of the anti-CD30 antibody-drug conjugate is administered via a second pharmaceutical composition. In some examples, the first pharmaceutical composition and the second pharmaceutical composition are administered concurrently. In some examples, the first pharmaceutical composition and the second pharmaceutical composition are administered sequentially. In some examples, the first pharmaceutical composition is administered at a higher dose than the second pharmaceutical composition. In some examples, the first pharmaceutical composition is administered at a lower dose than the second pharmaceutical composition.

In some examples, the genetically engineered CCR4-IL2 bispecific immunotoxin comprises an anti-human CCR4 scFv fused to a truncated diphtheria toxin DT390. In some examples, the genetically engineered CCR4-IL2 bispecific immunotoxin comprises a human IL2 peptide domain. In some examples, the therapeutically effective amount of the genetically engineered CCR4-IL2 bispecific immunotoxin and the therapeutically effective amount of the anti-CD30 antibody-drug conjugate is administered via a pharmaceutical composition. In some examples, the cutaneous T-cell lymphoma comprises CCR4⁺ and CD25⁺ cutaneous T-cell lymphoma. In some examples, the genetically engineered CCR4-IL2 bispecific immunotoxin reduces an amount of tumor-infiltrating Treg cells in the subject. In some examples, the CCR4-IL2 bispecific immunotoxin and the anti-CD30 antibody-drug conjugate are administered intravenously.

In some examples, methods may further involve performing a biopsy on a tumor within the subject. In some examples, administering the CCR4-IL2 bispecific immunotoxin and the anti-CD30 antibody-drug conjugate causes a reduction in one or more of a tumor volume, tumor weight, tumor number, or tumor metastasis.

In accordance with embodiments disclosed herein, a system for treating or alleviating at least one symptom of cutaneous T-cell lymphoma in a subject diagnosed with cutaneous T-cell lymphoma may include at least one injection device configured to administer to the subject a therapeutically effective amount of a first pharmaceutical composition comprising a genetically engineered CCR4-IL2 bispecific immunotoxin and a therapeutically effective amount of a second pharmaceutical composition comprising an anti-CD30 antibody-drug conjugate. In some examples, the genetically engineered CCR4-IL2 bispecific immunotoxin comprises an anti-human CCR4 scFv fused to a truncated diphtheria toxin DT390. In some examples, the genetically engineered CCR4-IL2 bispecific immunotoxin comprises a human IL2 peptide domain. In some examples, an amino acid sequence of the genetically engineered CCR4-IL2 bispecific immunotoxin is at least 90% identical to SEQ ID NO: 2. In some examples, the first pharmaceutical composition is administered at a higher dose than the second pharmaceutical composition. In some examples, the first pharmaceutical composition is administered at a lower dose than the second pharmaceutical composition. In some examples, the at least one injection device comprises an intravenous injection device.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Detailed Description and no limitation as to the scope of the present disclosure is intended by either the inclusion or non-inclusion of elements, components, etc. in this Summary. Other features and advantages of the invention will be apparent from the following Detailed Description and claims.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the Detailed Description of certain embodiments presented herein.

FIG. 1 is a schematic representation of a genetically engineered CCR4-1L2 bispecific immunotoxin (“CCR4-IL2 IT”) used in accordance with embodiments disclosed herein.

FIG. 2A is a panel showing flow cytometry binding affinity analysis of Alexa Fluor 488-labeled CCR4-IL2 IT or an anti-CD30 antibody-drug conjugate (brentuximab) with human CD25⁺CCR4⁺CD30⁺ Hut102/6TG cells. Fluorescein-mouse antihuman/rat CCR4 monoclonal antibody (“mAb”), fluorescein isothiocyanate (“FITC”)-mouse antihuman CD25 mAb, and phycoerythrin (“PE”)-mouse antihuman CD30 mAb were included as positive controls. The data are representative of three individual experiments.

FIG. 2B is a graph showing dissociation constants (“K_{D}”) for CCR4-IL2 IT determined using nonlinear regression analysis of the flow cytometry data with a saturation binding equation (GraphPad Prism 9.4.1). The median fluorescence intensity (“MFI”) was plotted over a wide range of concentrations of the Alexa Fluor 488-labeled CCR4-1L2 IT. Nonlinear regression analysis was based on the equation Y=Bₘₐₓ 9X/(KD+X), where Y=MFI at a given concentration of Alexa Fluor 488-labeled CCR4-IL2 IT after subtracting the background, X the concentration of the Alexa Fluor 488-labeled CCR4-1L2 IT, and Bₘₐₓ=the maximum specific binding in the same units as Y.

FIG. 2C is a graph showing dissociation constants (“K_{D}”) for the anti-CD30 antibody-drug conjugate determined using nonlinear regression analysis of the flow cytometry data with the same saturation binding equation (GraphPad Prism 9.4.1). The MFI was plotted over a wide range of concentrations of the Alexa Fluor 488-labeled anti-CD30 antibody-drug conjugate. Nonlinear regression analysis was based on the equation Y=Bₘₐₓ 9X/(K_{D}+X), where Y=MFI at a given concentration of Alexa Fluor 488-labeled anti-CD30 antibody-drug conjugate after subtracting the background, X=the concentration of the Alexa Fluor 488-labeled anti-CD30 antibody-drug conjugate, and Bₘₐₓ=the maximum specific binding in the same units as Y.

FIG. 3 is a graph showing the in vitro efficacy of CCR4-IL2 IT versus the anti-CD30 antibody-drug conjugate in the inhibition of the human CD25⁺CCR4⁺CD30⁺ T-cell lymphoma line, Hut102/6TG. The anti-CD30 antibody-drug conjugate data is represented by solid circles, CCR4-IL2 IT represented by triangles, a truncated diphtheria toxin based on a recombinant human IL2 fusion toxin that targets CD25+ CTCL (Ontak®-like IL2-IT (“IL2 IT”)) represented by squares, a C21 immunotoxin (“C21 IT”) as negative immunotoxin control represented by diamonds, and a genetically engineered, truncated diphtheria toxin-based recombinant single-chain fold-back diabody anti-human CCR4 immunotoxin (“CCR4 IT”) represented by empty circles. The y-axis shows the inhibition rate of cell viability calculated by determining the number of viable cells based on quantification of the ATP present. The x-axis shows the plated immunotoxin concentration. Cycloheximide (1.25 mg/mL⁻¹) was used as a positive control. The negative control consisted of cells without immunotoxin. Data are representative of three assays.

FIG. 4 is a graph showing an in vivo efficacy comparison of CCR4-IL2 IT versus the anti-CD30 antibody-drug conjugate in an immunodeficient mouse tumor model of CTCL. Days of survival are shown on the x-axis, and percent survival shown on the y-axis.

FIG. 5 is an image panel showing liver necropsy gross examination samples of representative tumor-bearing mice administered either CCR4-IL2 IT, CCR4 IT, IL2 IT, anti-CD30 antibody-drug conjugate (full dose), a combination of CCR4-IL2 IT and the anti-CD30 antibody-drug conjugate (full dose), C21 IT, or an anti-CD30 antibody-drug conjugate matching dose.

FIG. 6 is an image panel showing liver pathology analysis of representative tumor-bearing mice on day 28 of the experiment represented in FIGS. 4 and 5.

FIG. 7 is a flowchart showing a method of treating a subject afflicted with CTCL in accordance with embodiments disclosed herein.

### DEFINITIONS

Unless defined otherwise below, all technical and scientific terms used herein have the same meaning as commonly understood by one of ordinary skill in the art to which this disclosure belongs.

As used herein, the terms “treat,” “treating,” “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition or disorder. “Treatment” may refer to targeted therapy in some examples.

The terms “treat”, “treating” and “treatment” may further refer to eliminating, reducing, suppressing, or ameliorating, either temporarily or permanently, either partially or completely, a clinical symptom, manifestation or progression of an event, disease or condition associated with the oncological disorders and diseases described herein. As is recognized in the pertinent field, methods and drugs employed as therapies may reduce the severity of a given disease state, but need not abolish every manifestation of the disease to be regarded as useful. Similarly, a prophylactically administered treatment need not be completely effective in preventing the onset of a condition to constitute a viable prophylactic method or agent. Simply reducing the impact of a disease (for example, as disclosed herein, decreasing cancer cell growth rate, reducing tumor size, reducing tumor weight, reducing or preventing metastasis, etc. and/or reducing the number or severity of associated symptoms, or by increasing the effectiveness of another treatment, or by producing another beneficial effect), or reducing the likelihood that the disease will occur or worsen in a subject, is sufficient. One embodiment of the invention is directed to a method for determining the efficacy of treatment comprising administering to a patient therapeutic treatment in an amount, duration, and repetition sufficient to induce a sustained improvement over pre-existing conditions, or a baseline indicator that reflects the severity of the particular disorder.

“Reducing,” “reduce,” or “reduction” means decreasing the presence, severity, or duration of a cancer, a cancer symptoms, or cancerous tumors.

As used herein, “subject” means a human or other mammal. A subject can be considered in need of treatment. The disclosed compositions, methods, and systems may be effective to treat patients diagnosed with cancer or subjects at risk of developing cancer.

“Administration of” and “administering a” compound, composition, or agent should be understood to mean providing a compound, composition, or agent, a prodrug of a compound, composition, or agent, or a pharmaceutical composition as described herein. The compound, agent or composition may be provided or administered by another person to the subject (e.g., infusion) or it may be self-administered by the subject.

“Intravenous” administration refers to administering a drug (e.g., the disclosed CCR4-IL2 IT alone or together with an antibody-drug conjugate, and/or pharmaceutically acceptable forms thereof) into a vein of a patient, e.g., by infusion (slow therapeutic introduction into the vein).

Intraperitoneal administration or injection refers to administering a drug (e.g., the disclosed peptide and/or pharmaceutically acceptable forms thereof) into the peritoneum of a patient.

“Infusion” or “infusing” refers to the introduction of a drug-containing solution into the body through a vein for therapeutic purposes. Generally, this is achieved via an intravenous (IV) bag.

An “intravenous bag” or “IV bag” is a bag that can hold a solution which can be administered via the vein of a patient. In one embodiment, the solution is a saline solution (e.g. about 0.9% or about 0.45% NaCl). Optionally, the IV bag is formed from polyolefin or polyvinyl chloride.

By “co-administering” is meant intravenously administering two (or more) drugs during the same administration, rather than sequential infusions of the two or more drugs. Generally, this will involve combining the two (or more) drugs into the same IV bag prior to co-administration thereof.

The terms “active ingredient” or “active pharmaceutical ingredient” as used herein refer to a pharmaceutical agent, active ingredient, compound, or substance, compositions, or mixtures thereof, that provide a pharmacological, often beneficial, effect. In some embodiments, the active ingredient is CCR4-IL2 IT, which may be administered in combination with an anti-CD30 antibody-drug conjugate.

“Pharmaceutical compositions” or “pharmaceutical formulations” are compositions that include an amount (for example, a unit dosage) of one or more of the disclosed compounds, e.g., CCR4-IL2 IT, together with one or more non-toxic pharmaceutically acceptable additives, including carriers, diluents, and/or adjuvants, and optionally other biologically active ingredients. Such pharmaceutical compositions may be prepared by standard pharmaceutical formulation techniques such as those disclosed in Remington's Pharmaceutical Sciences, Mack Publishing Co., Easton, Pa. (19th Edition).

As used herein, a “pharmaceutically acceptable excipient” or a “pharmaceutically acceptable carrier” means a pharmaceutically acceptable material, composition, or vehicle that contributes to the desired form or consistency of the pharmaceutical composition. Each excipient or carrier must be compatible with other ingredients of the pharmaceutical composition when comingled such that interactions which would substantially reduce the efficacy of the compositions of this disclosure when administered to a subject and interactions which would result in pharmaceutical compositions that are not pharmaceutically acceptable are avoided. In addition, each excipient or carrier must be of sufficiently high purity to render it pharmaceutically acceptable. Non-limiting examples of pharmaceutically acceptable carriers may include lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methylhydroxybenzoate, propylhydroxybenzoate, talc, magnesium stearate, mineral oil or the like. In addition or alternatively, a carrier may include a lubricant, a wetting agent, a flavor, an emulsifier, a suspending agent, a preservative, or the like.

The agents, compounds, compositions, antibodies, etc. used in the implementations described herein are considered to be purified and/or isolated prior to use. Purified materials are typically “substantially pure,” meaning that CCR4-IL2 IT has been isolated from other components.

As used herein, the terms “identity” or “similarity” denote relationships between two or more polypeptide sequences or their underlying nucleic acid sequences, as determined by comparing the sequences. In the art, “identity” also means the degree of sequence relatedness between polypeptide or polynucleotide sequences, as determined by the match between strings of such sequences.

The term “amelioration” as used herein refers to any improvement of a disease state (for example cancer) of a patient, by the administration of one or more treatments and/or compositions, according to the present disclosure, to such patient or subject in need thereof. Such an improvement may be seen as a slowing down the progression or stopping the progression of the disease of the patient, and/or as a decrease in severity of disease symptoms, an increase in frequency or duration of disease symptom-free periods or a prevention of impairment or disability due to the disease.

An amino acid within the disclosed peptide compositions can be substituted to create an engineered CCR4-IL IT molecule. In some embodiments, the amino acids of the disclosed peptides may be natural, synthetic, for example derivatized native or non-native amino acids, for example diethyl lysine. The amino acid residue can be replaced by a residue having similar physiochemical characteristics, that is a ‘conservative substitution’—e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, for example based on size, charge, polarity, hydrophobicity, chain rigidity/orientation, etc., are well known in the art of protein engineering. Polypeptides comprising conservative amino acid substitutions can be tested in any one of the assays described herein to confirm that a desired activity, e.g. binding, specificity, and/or function of a native or reference polypeptide is achieved.

The terms “dosage” or “dose” as used herein denote any form of the active ingredient formulation that contains an amount sufficient to produce a therapeutic effect with a single administration.

The term “effective amount” refers to an amount of a compound of this disclosure or other active ingredient sufficient to provide a therapeutic or prophylactic benefit in the treatment or prevention of a disease or to delay or minimize symptoms associated with a disease. Further, a therapeutically effective amount with respect to a compound of this disclosure means that amount of therapeutic agent alone, or in combination with other therapies, that provides a therapeutic benefit in the treatment or prevention of a disease. Used in connection with a compound of the present disclosure, the term can encompass an amount that improves overall therapy, reduces or avoids symptoms or causes of disease, or enhances the therapeutic efficacy or synergies with another therapeutic agent.

The phrase “therapeutically effective amount” means an amount of a compound of the present disclosure that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (“TTP”) and/or determining the response rate (“RR”).

The term “mammal” includes, but is not limited to, humans, mice, rats, guinea pigs, monkeys, dogs, cats, horses, cows, pigs, and sheep.

A “patient” or “subject” includes an animal, such as a human, cow, horse, sheep, lamb, pig, chicken, turkey, quail, cat, dog, mouse, rat, rabbit or guinea pig. The animal can be a mammal such as a non-primate and a primate (e.g., monkey and human). In one embodiment, a patient is a human, such as a human infant, child, adolescent or adult.

In this description, a “pharmaceutically acceptable salt” is a pharmaceutically acceptable, organic or inorganic acid or base salt of a compound of this disclosure. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The term “prevention” as used herein means the avoidance of the occurrence or of the re-occurrence of a disease as specified herein, by the administration of an active compound, for example the disclosed peptide molecules alone or together with an antibody-drug conjugate, according to this disclosure to a subject in need thereof.

As used herein, the terms “protein” and “polypeptide” may be used interchangeably to designate a series of amino acid residues connected to each other by peptide bonds between the alpha-amino and carboxy groups of adjacent residues. The terms “protein” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” may be used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

“Subject in need”, “patient” or those “in need of treatment” include those already with existing disease (i.e., cancer, oncologic pathologies, CTCL, breast cancer, lung cancer, head and neck cancer, etc.), as well as those at risk of the disease. The terms also include human and other mammalian subjects that receive either prophylactic or therapeutic treatments as disclosed herein.

The term “specifically binds” is “antigen specific,” is “specific for,” “selective binding agent,” “specific binding agent,” “binding affinity” or is “selective” for a target refers to a peptide molecule (e.g., CCR4-IL2 IT) that binds a target protein (e.g., CCR4 or CD25) with greater affinity than other proteins, especially related proteins.

As used herein, the term “about” can mean relative to the recited value, e.g., amount, dose, temperature, time, percentage, etc., ±10%, ±9%, ±8%, ±7%, ±6%, ±5, ±4%, ±3%, ±2%, or ±1%.

The singular “a,” “an,” and “the” include plural referents unless context clearly indicates otherwise. Similarly, the word “or” is intended to include “and” unless the context clearly indicates otherwise. The term “comprises” means “includes” or “contains.” Also, “comprising A or B” means including A or B, or A and B, unless the context clearly indicates otherwise. It is to be further understood that all molecular weight or molecular mass values given for compounds are approximate, and are provided for description. Although methods and materials similar or equivalent to those described herein can be used in the practice or testing of this disclosure, suitable methods and materials are described below. In addition, the materials, methods, and examples are illustrative only and not intended to be limiting.

In the following sections, certain exemplary compositions and methods are described in order to detail certain embodiments of the invention. It will be understood by one skilled in the art that practicing the certain embodiments does not require the employment of all or even some of the specific details outlined herein, but rather that concentrations, times and other specific details can be modified through routine experimentation. In some cases, well-known methods or components have not been included in the description.

### DETAILED DESCRIPTION

The methods and compositions disclosed herein provide an effective targeted therapy for cancer, non-limiting examples of which include cutaneous T-cell lymphoma (“CTCL”).

The disclosed methods of cancer treatment may involve administering to a subject afflicted with cancer, e.g., CTCL, a genetically engineered CCR4-IL2 bispecific immunotoxin (“CCR4-IL2 IT”) alone or together with at least one additional therapeutic agent, such as an anti-CD30 antibody-drug conjugate (hereinafter “anti-CD30 drug-antibody conjugate” or “antibody-drug conjugate” or “conjugate”). A pharmaceutical composition containing CCR4-IL2 IT may thus be administered alone or together with one or more additional pharmaceutical compositions or agents including, for example, those comprising an antibody-drug conjugate configured to target and deplete cells expressing certain cell membrane proteins, such as CD30. In some examples, administering CCR4-IL2 IT, alone, may be more effective in treating cancer than administering the anti-CD30 antibody-drug conjugate, alone. When administered in combination, e.g., concurrently or in succession, CCR4-IL2 IT and the anti-CD30 antibody-drug conjugate may exhibit a synergistic effect that is greater than the therapeutic effect achieved by either agent alone.

Embodiments may treat CTCL by, at least in part, targeting and depleting CCR4⁺ and/or CD25⁺ cancer cells, including CCR4⁺ and/or CD25⁺ tumor-infiltrating effector regulatory T-cells (“Treg cells”), which may enhance the overall treatment effect and provide a powerful adjuvant effect for broad-spectrum cancer treatment. Embodiments may also treat CTCL by, at least in part, targeting and depleting CD30⁺ cancer cells. Accordingly, the therapeutic effects disclosed herein may be employed to combat a variety of cancer types, in addition to CTCL, some of which may be exacerbated or associated with an immunosuppressive tumor microenvironment. Certain non-limiting examples may involve the treatment of CTCL, specifically.

Effective cancer treatment achieved by implementing embodiments of the present disclosure may be evidenced by reductions in tumor size (including mass and dimensions), tumor number, tumor metastasis, and/or tumor growth, as well as reductions in the severity and size of skin rashes, skin lesions, and/or abnormal skin growths, along with a reduction in the number and/or proliferation of cancer cells in a subject. Effective treatment may also comprise prolonged survival of subjects afflicted with cancer. One or more of these effects may be accompanied by a reduction or elimination of one or more symptoms associated with cancer.

### CCR4-IL2 Bispecific Immunotoxin and Antibody-Drug Conjugate

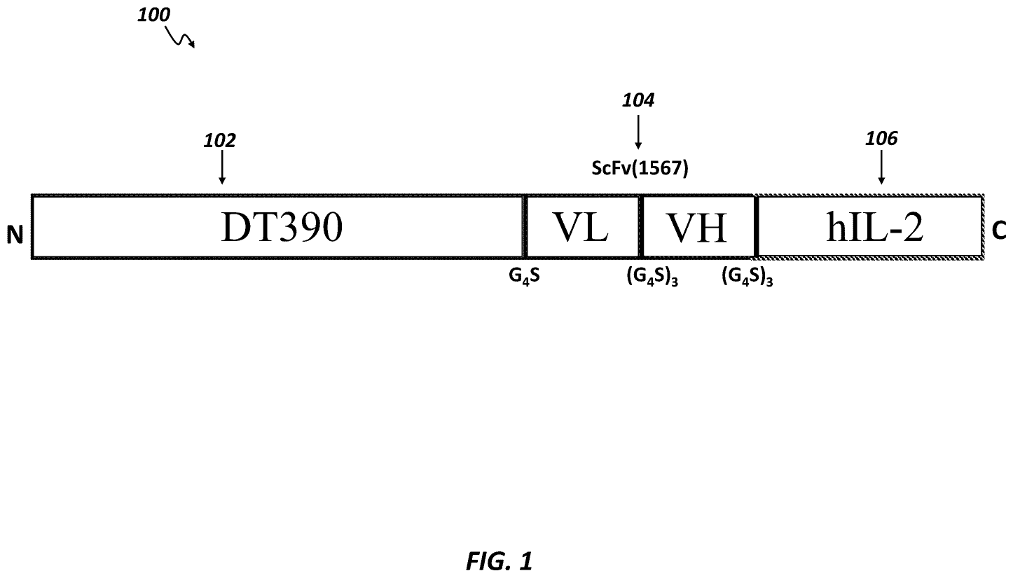

Embodiments of CCR4-IL2 IT, represented in FIG. 1 as CCR4-IL2 IT 100, may include a truncated diphtheria toxin 102 fused to one anti-human CCR4 single-chain variable fragment (“scFv”) 104, which is fused to the cytokine human interleukin-2 (“IL2”) 106 at the C-terminus. The genetically engineered, CCR4-IL2 bispecific immunotoxin thus targets surface markers C-C chemokine receptor 4 (“CCR4”) and/or IL-2 receptor α-chain (“CD25”). As further shown in FIG. 1, the specific toxin at the N-terminus may be truncated diphtheria toxin DT390. Additional details of CCR4-IL2 IT, and variations thereof, are described in US 2022/0127368 A1, the entire contents of which are incorporated by reference herein.

Altogether, CCR4-IL2 IT may be about 86 kDa in some embodiments. Additional variations of the recombinant protein may vary in size, ranging from about 76 kDa to about 96 kDa, or any value therebetween, including about 77 kDa, about 78 kDa, about 79 kDa, about 80 kDa, about 81 kDa, about 82 kDa, about 83 kDa, about 84 kDa, about 85 kDa, about 87 kDa, about 88 kDa, about 89 kDa, about 90 kDa, about 91 kDa, about 92 kDa, about 93 kDa, about 94 kDa, or about 95 kDa.

CCR4-IL2 IT was developed using a novel, advanced diphtheria toxin-resistant yeast (Pichia pastoris) expression system, as previously described (Wang et al., Bioconjug Chem. 22, 2014-2020, 2011; Example 1 and Peraino, J S et al., J. Immunol. Methods 398-399, 33-43, 2013). The yeast expression system overcomes expression and purification problems encountered using E. coli-based expression systems to deliver high production level and excellent purification quality of CCR4-IL2 IT.

Embodiments of CCR4-IL2 IT may have nucleotide sequence at least about 90%, 95%, 96%, 97%, 98%, 99%, or 100% identical to SEQ ID NO: 1, which encodes a human CCR4-IL2 IT having SEQ ID NO: 2.

SEQ ID NO: 1:
gctggtgctg acgacgtcgt cgactcctcc aagtccttcg tcatggagaa cttcgcttcc
taccacggga ccaagccagg ttacgtcgac tccatccaga agggtatcca gaagccaaag
tccggcaccc aaggtaacta cgacgacgac tggaaggggt tctactccac cgacaacaag
tacgacgctg cgggatactc tgtagataat gaaaacccgc tctctggaaa agctggaggc
gtggtcaagg tcacctaccc aggtctgact aaggtcttgg ctttgaaggt cgacaacgct
gagaccatca agaaggagtt gggtttgtcc ttgactgagc cattgatgga gcaagtcggt
accgaagagt tcatcaagag attcggtgac ggtgcttcca gagtcgtctt gtccttgcca
ttcgctgagg gttcttctag cgttgaatat attaataact gggaacaggc taaggctttg
tctgttgaat tggagattaa cttcgaaacc agaggtaaga gaggtcaaga tgcgatgtat
gagtatatgg ctcaagcctg tgctggtaac agagtcagac gttctgttgg ttcctctttg
tcctgtatca acctagactg ggacgtcatc agagacaaga ctaagaccaa gatcgagtct
ttgaaagagc atggcccaat caagaacaag atgtccgaat cccccgctaa gaccgtctcc
gaggaaaagg ccaagcaata cctagaagag ttccaccaaa ccgccttgga gcatcctgaa
ttgtcagaac ttaaaaccgt tactgggacc aatcctgtat tcgctggggc taactatgcg
gcgtgggcag taaacgttgc gcaagttatc gatagcgaaa cagctgataa tttggaaaag
acaactgctg ctctttcgat acttcctggt atcggtagcg taatgggcat tgcagacggt
gccgttcacc acaatacaga agagatagtg gcacaatcca tcgctttgtc ctctttgatg
gttgctcaag ctatcccatt ggtcggtgag ttggttgaca tcggtttcgc tgcctacaac
ttcgtcgagt ccatcatcaa cttgttccaa gtcgtccaca actcctacaa ccgtccggct
tactccccag gtcacaagac ccaaccattc ttgccatggg gtggtggtgg ttctgacatt
gagttgactc aatctccatc ttccttggct gtttctgctg gtgagaaggt tactatgtct
tgtaagtctt cccaatctat tttgtactct tccaaccaaa agaactactt ggcttggtac
caacaaaagc caggtcaatc tccaaagttg ttgatttact gggcttctac tagagagtct
ggtgttccag acagattcac tggttctggt tctggtactg acttcacttt gactatttct
tccgttcaag ctgaggactt ggctgtttac tactgtcacc aatacttgtc ttcctacact
ttcggtggtg gtactaagtt ggagattaag ggtggtggtg gttctggtgg tggtggatct
ggtggtggtg gttctcaagt tcaattgcaa caatctggtc cagagttggt tagaccaggt
gcttctgtta gaatttcttg taaggcttct ggttacactt tcgcttctta ctacattcaa
tggatgaagc aaagaccagg tcaaggtttg gagtggattg gttggattaa cccaggtaac
gttaacacta agtacaacga gaagttcaag ggtaaggcta ctttgactgc tgacaagtct
tccactaccg cttacatgca attgtcttcc ttgacttctg aggactctgc tgtttacttc
tgtgctagat ccacttacta cagaccattg gactactggg gtcaaggtac taccgttact
gtttcttccg gtggtggtgg ttctggtggt ggtggatccg gtggtggtgg ttctgctcca
acttcttctt ctactaagaa gactcaattg caattggagc acttgttgtt ggacttgcaa
atgattttga acggtattaa caactacaag aacccaaagt tgactagaat gttgactttc
aagttctaca tgccaaagaa ggctactgag ttgaagcact tgcaatgttt ggaggaggaa
ttgaagccat tggaggaagt tttgaacttg gctcaatcta agaacttcca cttgagacca
agagacttga tttctaacat taacgttatt gttttggagt tgaagggttc tgagactact
ttcatgtgtg agtacgctga cgagactgct actattgttg agttcttgaa cagatggatt
actttctgtc aatctattat ctctactttg actcaccacc accaccacca c
SEQ ID NO: 2:
Ala Gly Ala Asp Asp Val Val Asp Ser Ser Lys Ser Phe Val Met Glu
Asn Phe Ala Ser Tyr His Gly Thr Lys Pro Gly Tyr Val Asp Ser Ile
Gln Lys Gly Ile Gln Lys Pro Lys Ser Gly Thr Gln Gly Asn Tyr Asp
Asp Asp Trp Lys Gly Phe Tyr Ser Thr Asp Asn Lys Tyr Asp Ala Ala
Gly Tyr Ser Val Asp Asn Glu Asn Pro Leu Ser Gly Lys Ala Gly Gly
Val Val Lys Val Thr Tyr Pro Gly Leu Thr Lys Val Leu Ala Leu Lys
Val Asp Asn Ala Glu Thr Ile Lys Lys Glu Leu Gly Leu Ser Leu Thr
Glu Pro Leu Met Glu Gln Val Gly Thr Glu Glu Phe Ile Lys Arg Phe
Gly Asp Gly Ala Ser Arg Val Val Leu Ser Leu Pro Phe Ala Glu Gly
Ser Ser Ser Val Glu Tyr Ile Asn Asn Trp Glu Gln Ala Lys Ala Leu
Ser Val Glu Leu Glu Ile Asn Phe Glu Thr Arg Gly Lys Arg Gly Gln
Asp Ala Met Tyr Glu Tyr Met Ala Gln Ala Cys Ala Gly Asn Arg Val
Arg Arg Ser Val Gly Ser Ser Leu Ser Cys Ile Asn Leu Asp Trp Asp
Val Ile Arg Asp Lys Thr Lys Thr Lys Ile Glu Ser Leu Lys Glu His
Gly Pro Ile Lys Asn Lys Met Ser Glu Ser Pro Ala Lys Thr Val Ser
Glu Glu Lys Ala Lys Gln Tyr Leu Glu Glu Phe His Gln Thr Ala Leu
Glu His Pro Glu Leu Ser Glu Leu Lys Thr Val Thr Gly Thr Asn Pro
Val Phe Ala Gly Ala Asn Tyr Ala Ala Trp Ala Val Asn Val Ala Gln
Val Ile Asp Ser Glu Thr Ala Asp Asn Leu Glu Lys Thr Thr Ala Ala
Leu Ser Ile Leu Pro Gly Ile Gly Ser Val Met Gly Ile Ala Asp Gly
Ala Val His His Asn Thr Glu Glu Ile Val Ala Gln Ser Ile Ala Leu
Ser Ser Leu Met Val Ala Gln Ala Ile Pro Leu Val Gly Glu Leu Val
Asp Ile Gly Phe Ala Ala Tyr Asn Phe Val Glu Ser Ile Ile Asn Leu
Phe Gln Val Val His Asn Ser Tyr Asn Arg Pro Ala Tyr Ser Pro Gly
His Lys Thr Gln Pro Phe Leu Pro Trp Gly Gly Gly Gly Ser Asp Ile
Glu Leu Thr Gln Ser Pro Ser Ser Leu Ala Val Ser Ala Gly Glu Lys
Val Thr Met Ser Cys Lys Ser Ser Gln Ser Ile Leu Tyr Ser Ser Asn
Gln Lys Asn Tyr Leu Ala Trp Tyr Gln Gln Lys Pro Gly Gln Ser Pro
Lys Leu Leu Ile Tyr Trp Ala Ser Thr Arg Glu Ser Gly Val Pro Asp
Arg Phe Thr Gly Ser Gly Ser Gly Thr Asp Phe Thr Leu Thr Ile Ser
Ser Val Gln Ala Glu Asp Leu Ala Val Tyr Tyr Cys His Gln Tyr Leu
Ser Ser Tyr Thr Phe Gly Gly Gly Thr Lys Leu Glu Ile Lys Gly Gly
Gly Gly Ser Gly Gly Gly Gly Ser Gly Gly Gly Gly Ser Gln Val Gln
Leu Gln Gln Ser Gly Pro Glu Leu Val Arg Pro Gly Ala Ser Val Arg
Ile Ser Cys Lys Ala Ser Gly Tyr Thr Phe Ala Ser Tyr Tyr Ile Gln
Trp Met Lys Gln Arg Pro Gly Gln Gly Leu Glu Trp Ile Gly Trp Ile
Asn Pro Gly Asn Val Asn Thr Lys Tyr Asn Glu Lys Phe Lys Gly Lys
Ala Thr Leu Thr Ala Asp Lys Ser Ser Thr Thr Ala Tyr Met Gln Leu
Ser Ser Leu Thr Ser Glu Asp Ser Ala Val Tyr Phe Cys Ala Arg Ser
Thr Tyr Tyr Arg Pro Leu Asp Tyr Trp Gly Gln Gly Thr Thr Val Thr
Val Ser Ser Gly Gly Gly Gly Ser Gly Gly Gly Gly Ser Gly Gly Gly
Gly Ser Ala Pro Thr Ser Ser Ser Thr Lys Lys Thr Gln Leu Gln Leu
Glu His Leu Leu Leu Asp Leu Gln Met Ile Leu Asn Gly Ile Asn Asn
Tyr Lys Asn Pro Lys Leu Thr Arg Met Leu Thr Phe Lys Phe Tyr Met
Pro Lys Lys Ala Thr Glu Leu Lys His Leu Gln Cys Leu Glu Glu Glu
Leu Lys Pro Leu Glu Glu Val Leu Asn Leu Ala Gln Ser Lys Asn Phe
His Leu Arg Pro Arg Asp Leu Ile Ser Asn Ile Asn Val Ile Val Leu
Glu Leu Lys Gly Ser Glu Thr Thr Phe Met Cys Glu Tyr Ala Asp Glu
Thr Ala Thr Ile Val Glu Phe Leu Asn Arg Trp Ile Thr Phe Cys Gln
Ser Ile Ile Ser Thr Leu Thr His His His His His His

Examples of CCR4-IL2 IT may also include one or more linker sequences, as further described in US 2022/0127368.

The antibody-drug conjugate may comprise a mouse human chimeric anti-CD30 monoclonal antibody (“mAb”) conjugated to the anti-mitotic agent, monomethyl auristatin E (“MMAE”), with a protease-sensitive peptide linker. The conjugate is selectively delivered to CD30⁺ cells, where the cytotoxic compound is released intracellularly and causes cell-cycle arrest by interfering with microtubule formation. The antibody-drug conjugate may be or include brentuximab vedotin in some examples.

### Pharmaceutical Compositions

The pharmaceutical compositions of this disclosure may be suitable for treating a variety of cancers, including CTCL. Embodiments of the pharmaceutical composition may include a pharmaceutically effective amount of CCR4-IL2 IT. The pharmaceutical composition may also include a pharmaceutically acceptable carrier configured to facilitate and/or stabilize CCR4-IL2 IT during and beyond delivery to the target site(s) of a subject.

In some examples, a pharmaceutical composition administered to a subject may include a therapeutically effective amount of an antibody-drug conjugate, such as the disclosed anti-CD30 antibody-drug conjugate. Such a pharmaceutical composition may include a pharmaceutically acceptable carrier configured to facilitate and/or stabilize the antibody-drug conjugate during and beyond delivery to the target site(s) of a subject.

Examples may involve co-administering one pharmaceutical composition containing CCR4-IL2 IT and another pharmaceutical composition containing the antibody-drug conjugate. Examples may involve administering a single pharmaceutical composition containing CCR4-IL2 IT and the antibody-drug conjugate.

In embodiments, the pharmaceutical composition(s) may include or be administered concurrently with one or more excipients. Suitable excipients may vary depending upon the particular dosage form utilized. In addition, suitable excipients may be chosen for a particular function, such as the ability to facilitate the production of stable dosage forms. Excipients may also be chosen for regulatory compliance. Non-limiting excipient examples include: fillers, binders, disintegrants, lubricants, glidants, granulating agents, coating agents, wetting agents, solvents, co-solvents, suspending agents, emulsifiers, coloring agents, anticaking agents, humectants, chelating agents, plasticizers, viscosity agents, antioxidants, preservatives, stabilizers, and surfactants. In some embodiments, one or more delivery vehicles may be utilized, non-limiting examples of which may include nanoparticles, nanogels, and/or adeno-associated viral vectors (“AAV vectors”). The skilled artisan will appreciate that certain pharmaceutically acceptable excipients may serve more than one function and may serve alternative functions depending on how much of the excipient is present in the final composition and which other ingredients are present in the composition.

In embodiments, the CCR4-IL2 IT protein (and antibody-drug conjugate) may be administered concurrently with one or more buffering agents and/or diluents, non-limiting examples of which may include various concentrations of sodium hydroxide and sodium phosphate.

### Therapeutic Approaches

Methods of treating a cancer may involve administering to a subject a therapeutically effective amount of one or more pharmaceutical compositions containing CCR4-IL2 IT alone or in combination with a therapeutically effective amount of an anti-CD30 antibody-drug conjugate, non-limiting examples of which may include brentuximab. The composition(s) may be administered after a cancer is diagnosed, for example after performing a biopsy. In some examples, prophylactic administration may be performed after determining that a subject is exhibiting signs of a cancer, such as a tumor or skin lesion. A disclosed pharmaceutical composition may be infused or injected into a subject soon after the subject is diagnosed with cancer. A broad spectrum of cancer types may be effectively treated via administration of CCR4-IL2 IT alone or together with the anti-CD30 antibody-drug conjugate, non-limiting examples of which may include CTCL.

Administration of a disclosed pharmaceutical composition may deplete CCR4⁺ and/or CD25⁺ CTCL cells, as well as CCR4⁺ and/or CD25⁺ tumor-infiltrating effector Treg cells to enhance the overall effect of treatment. Administration of CCR4-IL2 IT may also effectively treat subjects with CD30⁻ and brentuximab-relapsed CTCL, and it may provide a combination treatment together with the anti-CD30 antibody-drug conjugate for subjects with CD30⁺CCR4⁺ and/or CD25⁺ CTCL.

The frequency of administration of the pharmaceutical compositions disclosed herein may vary. In embodiments, a pharmaceutically effective amount of the composition(s) may be administered daily, weekly, monthly, or yearly. The number of times the disclosed compositions are administered to a subject, along with the length of the treatment period, may depend on the severity or type of the cancerous condition. The length of the treatment period may also be patient-specific and re-evaluated periodically by a physician or other health care provider. In various embodiments, a pharmaceutical composition may be administered immediately following a cancer diagnosis, such as within one, two, six, 12, or 24 hours, or within one, two, three, four, five, six, seven days or more, including one or more weeks or months. The formulations may be administered once or multiple times, for example two, three, four, five, six, seven, eight, nine, ten times, or more. The pharmaceutical composition(s) may be administered to a subject for a predefined period of time, or until the condition is effectively treated, for example when all tumors have been depleted and/or removed, or when cancer cell proliferation has stopped or reversed.

As noted in the preceding section, methods of treatment may also involve administering to a subject a therapeutically effective amount of a pharmaceutical composition containing an antibody conjugated to a drug, the resulting conjugate configured to target the CD30 cell membrane protein and deplete CD30⁺ cells. A non-limiting example of such a composition may comprise brentuximab, and it may be co-administered or otherwise administered in combination with CCR4-IL2 IT to a subject afflicted with CTCL. Administration of the CCR4-IL2 IT pharmaceutical composition and the antibody-drug conjugate pharmaceutical composition may involve administering the compositions simultaneously, sequentially, or intermittently. The compositions may be administered at the same or different frequency. For example, a CCR4-IL2 IT pharmaceutical composition may be administered to a subject more frequently than the antibody-drug conjugate pharmaceutical composition. In various non-limiting embodiments, a CCR4-IL2 IT pharmaceutical composition may be administered daily, while the antibody-drug conjugate pharmaceutical composition may also be administered daily, or once every other day, once every three days, once every four days, once every five days, once every 6 days, once every week, once every two weeks, once every three weeks, once every month, etc. Co-administration of pharmaceutical compositions containing CCR4-IL2 IT and the antibody-drug conjugate, together or separately, may result in a synergistic therapeutic effect, such that treatment of the subject afflicted with cancer is more effective than when either pharmaceutical composition is administered alone. Therapeutic synergy may be evidenced by more significant reductions in skin lesions, tumor size, metastasis, and/or growth, along with a decrease in cancerous cells, resulting increases in subject survival and quality of life. Such therapeutic effects may also be achieved after administration of CCR4-IL2 IT or the antibody-drug conjugate, but to a lesser extent in some examples relative to the combination treatment.

Embodiments of each pharmaceutical composition may include, or be administered concurrently with, at least one pharmaceutically acceptable carrier. Relatedly, the pharmaceutical composition may be administered singly or in combination with other therapeutic agents, either serially or simultaneously. Such additional agents may or may not be formulated to treat the same conditions.

The pharmaceutical compositions disclosed herein may be administered using an infusion system or device, or an injection device, such as tuberculin syringe or an IV drip device, which may be configured specifically for the purposes described herein. In some examples, the administration device may be a single-use device, which may be included in a kit that also includes a single dose of a pharmaceutical composition. Accordingly, an injection device may constitute a part of a system for treating, reducing the risk of, preventing, or alleviating at least one symptom of retinal damage.

The therapeutically effective amount of a pharmaceutical composition administered to a subject may vary. In embodiments, a disclosed pharmaceutical composition containing CCR4-IL2 IT may be administered at a variety of doses ranging from about 5 μg/kg to about 50 μg/kg, about 10 μg/kg to about 40 μg/kg, about 15 μg/kg to about 30 μg/kg, about 20 μg/kg to about 25 μg/kg, or any level therebetween, including for example about 10 μg/kg, 12 μg/kg, 14 μg/kg, 16 μg/kg, 18 μg/kg, 20 μg/kg, 22 μg/kg, 24 μg/kg, 26 μg/kg, 28 μg/kg, or about 30 μg/kg. In some examples, the dose may not exceed about 30 μg/kg to avoid or minimize undesired off-target effects caused by CCR4-IL2 IT. Dosing may depend, for example, on the condition treated, the severity of the condition, the nature of the formulation (e.g., with or without a second pharmaceutical agent, e.g., an antibody-drug conjugate), the method of administration, the condition of the subject, the age of the subject, the weight of the subject, or combinations thereof. Dosage levels are typically sufficient to achieve a concentration at the site of action that is at least the same as a concentration that has been shown to be active in vitro, in vivo, or in tissue culture.

The therapeutically effective amount of a pharmaceutical composition containing an antibody-drug conjugate may also vary, and may be substantially the same or different than the therapeutically effective amount of a pharmaceutical composition containing CCR4-IL2 IT. In some examples, the antibody-drug conjugate may be administered at a variety of doses ranging from about 0.5 mg/kg to about 5 mg/kg, or about 0.8 mg/kg to about 4 mg/kg, or about 1 mg/kg to about 3 mg/kg, or any dose therebetween, including about 1.2 mg/kg, 1.4 mg/kg, 1.6 mg/kg, 1.8 mg/kg, 2 mg/kg, 2.2 mg/kg, 2.4 mg/kg, 2.6 mg/kg, or 2.8 mg/kg. The pharmaceutical composition may be administered as an intravenous infusion on a regular basis, for example as a 30-minute infusion every three weeks. The dosing schedule may vary, such that each dose of one or more pharmaceutical compositions is administered approximately weekly, biweekly, every three weeks, four weeks, five weeks, or more, or any period therebetween, with periodic evaluations and adjustments made as necessary. Dosing may also be determined on a patient-specific basis, such that dosing may be adjusted for patients having different weights. In one non-limiting embodiment, the antibody-drug conjugate may be administered at a dose of about 1.8 mg/kg as an intravenous infusion over about 30 minutes approximately every three weeks.

To accommodate multiple administration techniques and schedules, the pharmaceutical compositions disclosed herein may be prepared in a unit-dosage form or multiple-dosage form, along with a pharmaceutically acceptable carrier and/or excipient according to a method employed by those skilled in the art. Example formulations may be in the form of an aqueous or oil-based solution, a suspension, or an emulsion. For increased stability and long-term storage, the pharmaceutical compositions may be lyophilized.

### Kits

This disclosure further relates to a kit comprising one or more pharmaceutical compositions disclosed herein for use in a method of treating or alleviating a symptom of a cancer, such as CTCL. In certain embodiments, kits are provided for storage, transport and use in treating or alleviating a target disease, such as a cancer, as described herein. In some embodiments, kits can include one or more containers.

### EXAMPLES

The following examples are included to illustrate certain embodiments. It should be appreciated by those of skill in the art that the techniques disclosed in the examples represent techniques discovered to function well in the practice of the claimed methods, compositions and systems. However, those of skill in the art should, in light of the present disclosure, appreciate that changes can be made in some embodiments which are disclosed and still obtain a like or similar result without departing from the spirit and scope of embodiments of this disclosure.

Statistical analysis for the following examples involved determining the half maximal inhibitory concentration (IC₅₀) using nonlinear regression analysis (GraphPad Prism 9.4.0, GraphPad Software, San Diego, CA, USA). Kaplan-Meier survival curves were generated for the survival functions of the various treatment groups for the in vivo experiments. The primary comparison for the survival experiments was between CCR4-IL2 IT and the anti-CD30 antibody-drug conjugate (brentuximab) full-dose. The anti-CD30 antibody-drug conjugate is referred to in the following examples primarily as “conjugate” for ease of reference, only. Secondary comparisons included a comparison of the combination of CCR4-1L2 IT and the conjugate versus the conjugate, alone, as well as a comparison of the combination of CCR4-IL2 IT and the conjugate versus CCR4-IL2 IT, alone. Treatment effects on overall survival were estimated using a frailty Cox proportional hazard regression model, where the measures from the two sets of experiments in this study were combined, and a random effect was included in the model to account for the potential correlations among measures within each set of the experiments. Regarding sample size/power considerations, the experiments were powered on the primary comparison between CCR4-1L2 IT and the conjugate full-dose. Seven mice per group provided 80% power to detect a hazard ratio of 0.26 in favor of CCR4-IL2 IT using a one-sided test at a 0.05 significance level. All mice were followed until euthanasia. The p-values for survival curve comparisons were calculated using a Mantel-Cox log-rank test (GraphPad Prism 9.4.1). P<0.05 was considered statistically significant. SAS software 9.4 (SAS institute Inc. NC. USA) was used to carry out the frailty Cox proportional hazard regression analysis.

In the following description, CCR4 IT refers to a diphtheria toxin-based recombinant fold-back diabody anti-human CCR4 immunotoxin. 1L2-IT refers to an Ontak®-like IL2 immunotoxin, and C21 IT refers to a truncated diphtheria toxin-based immunotoxin.

### Example 1

The in vitro efficacy of CCR4-IL2 IT and the anti-CD30 antibody-drug conjugate (brentuximab) for treating CTCL was evaluated via multiple experiments. First, the binding affinity of CCR4-IL2 IT versus the conjugate to a human CTCL cell line was analyzed via flow cytometry. Specifically, human CD25⁺CCR4⁺CD30⁺ Hut102/6TG cells were stained with Alexa Fluor 488-labeled CCR4-IL2 IT or the conjugate at a wide range of concentrations (0.1 to 200 nM). Fluorescein-mouse anti-human/rat CCR4 mAb (26 nM), FITC-mouse anti-human CD25 mAb (50 nM), and PE-mouse anti-human CD30 mAb (25 nM) were included as positive controls. Unstained cells were used as a negative control. Flow cytometry was carried out using a CytoFLEX Flow cytometer (Beckman Coulter, Brea, CA, USA), and data was analyzed using FlowJo software (Flowjo, LLC, Ashland, OR, USA).

Dissociation constants (“K_{D}”) were determined using nonlinear regression analysis of the flow cytometry data with a saturation binding equation (GraphPad Prism 9.4.1). The median fluorescence intensity (“MFI”) was plotted versus the concentrations of the Alexa Fluor 488-labeled conjugate or CCR4-IL2 IT. Nonlinear regression analysis was based on the equation Y=Bmax×X/(K_{D}+X), where Y=MFI at a given concentration of Alexa Fluor 488-labeled conjugate or CCR4-IL2 IT after subtracting the background, X=the concentration of the Alexa Fluor 488-labeled conjugate or CCR4-IL2 IT, and Bmax=the maximum specific binding in the same units as Y.

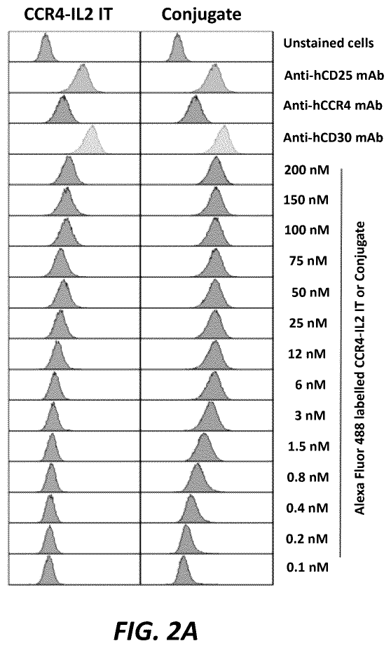

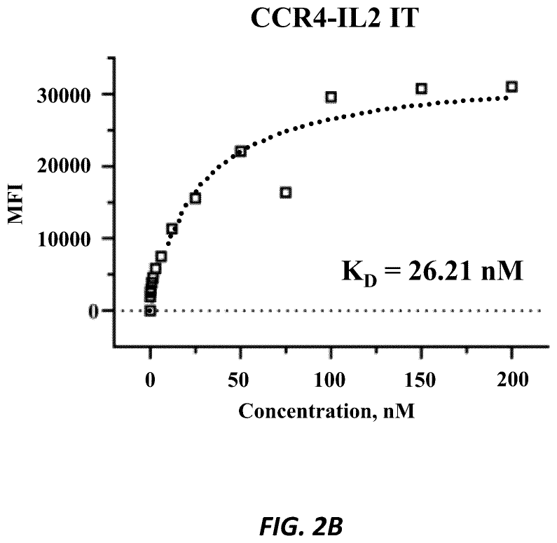

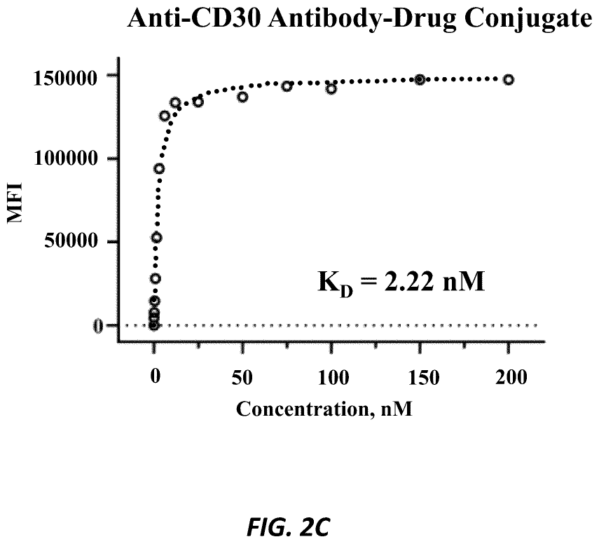

As shown in FIGS. 2A, 2B, and 2C, the in vitro binding affinity data demonstrated that both CCR4-IL2 IT and the conjugate bound to the CTCL cells, with the conjugate (K_{D}=2.22 nM) binding more strongly than CCR4-1L2 IT (K_{D}=22.61 nM).

A comparison of the in vitro efficacy of the conjugate versus CCR4-IL2 IT in the inhibition of the viability of human CD25⁺CCR4⁺CD30⁺ CTCL Hut102/6TG cells was then performed using a CellTiter-Glo® Luminescent Cell Viability Assay (Promega, Madison, WI, USA), which measured the luminescence produced by ATP production from metabolically active cells. Luminescence signals were measured using a BioTek Synergy LX Multi-Mode Reader.

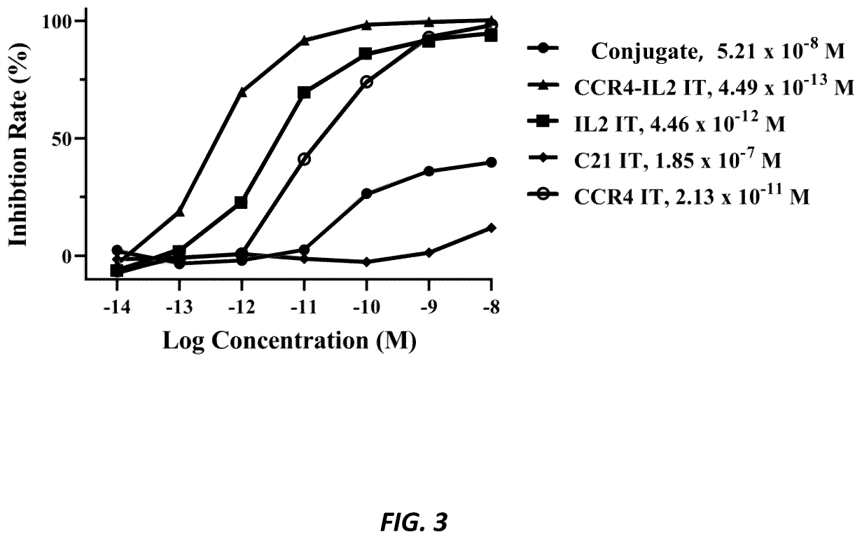

As shown in FIG. 3, increasing concentrations of the conjugate and CCR4-IL2 IT induced cell death, evidenced by a corresponding reduction in ATP-related fluorescence. Compared to each other, the data also showed that CCR4-IL2 IT (IC₅₀=4.49×10⁻¹³ M) was significantly more potent than the anti-CD30 antibody-drug conjugate (IC₅₀=5.21×10⁻⁸ M). Ontak®-like IL2 IT (IC₅₀=4.46×10⁻¹² M) and CCR4 IT (IC₅₀=2.13×10⁻¹¹ M) were also included as monospecific immunotoxin controls. C21 IT (IC₅₀=1.85×10⁻⁷ M) was included as a negative immunotoxin control.

Accordingly, while both were effective in depleting CTCL cells, the in vitro efficacy of CCR4-IL2 IT was significantly greater than the efficacy of the anti-CD30 antibody-drug conjugate in depleting CTCL cells.

### Example 2

In a second example, CCR4-IL2 IT was evaluated in vivo for its efficacy in treating CTCL via targeted therapy in an immunodeficient NSG mouse model of CTCL. As shown below in Table 1, NSG mice ranging from six to eight weeks old were divided into seven treatment (and control) groups: 1) CCR4-IL2 IT group (n=7); 2) CCR4 IT as a monospecific immunotoxin control targeting the CCR4 receptor group (n=7); 3) IL2 IT as another monospecific immunotoxin control targeting CD25 group (n=7); 4) anti-CD30 antibody-drug conjugate full dose group (n=7); 5) combination treatment with CCR4-IL2 IT and the conjugate full-dose group (n=7); 6) C21 IT as a negative control group (n=7); and 7) conjugate matching dose (exactly the same molar dose as the immunotoxins) group (n=7). All animals were intravenously injected on day 0 with 1.0×10⁷ human CD25⁺CCR4⁺CD30⁺ Hut102/6TG tumor cells via tail veins. The immunotoxin (CCR4-IL2 IT, or CCR4 IT, or IL2 IT, or C21 IT) or conjugate-matching doses were intraperitoneally (IP) injected starting on day 4 at 8.43×10⁻¹⁰ moles/kg, continuing once daily for ten consecutive days (ten doses total). A conjugate full-dose was IP injected starting on day 4 at 3 mg/kg, continuing once every other day for ten consecutive days (five doses total). For combination treatment, CCR4-IL2 IT was IP injected at 8.43×10⁻¹⁰ moles/kg once daily for ten consecutive days (ten doses in total), and a conjugate full-dose was IP injected at 3 mg/kg once every other day for ten days (five doses total). Tumor-bearing mice were observed daily for signs and symptoms of illness and scored at least twice weekly. The animals were humanely euthanized when the body condition score exceeded the limit, or the animal lost more than 15% of its pre-injection body weight.

TABLE 1

| | Mice | Therapeutic | Dose | Molar dose | Drug IP Injection Day (post-tumor cell injection) | | | | | | | | | | |
|---|---|---|---|---|---|---|---|---|---|---|---|---|---|---|---|
| Group | No. | Agent | (μg/kg) | (molar/kg) | 0 | 4 | 5 | 6 | 7 | 8 | 9 | 10 | 11 | 12 | 13 |
| CCR4- | 7 | CCR4-IL2 IT | 73 | 8.43E−10 | CTCL | X | X | X | X | X | X | X | X | X | X |
| IL2 IT | | | | | | | | | | | | | | | |
| CCR4 IT | 7 | CCR4 IT | 81.2 | 8.43E−10 | CTCL | X | X | X | X | X | X | X | X | X | X |
| IL2 IT | 7 | IL2 IT | 50 | 8.43E−10 | CTCL | X | X | X | X | X | X | X | X | X | X |
| Conjugate | 7 | conjugate | 3,000 | 1.96E−08 | CTCL | X | | X | | X | | X | | X | |
| full dose | | | | | | | | | | | | | | | |
| Combined | 7 | CCR4-IL2 IT | 73 | 8.43E−10 | CTCL | X | X | X | X | X | X | X | X | X | X |
| treatment | | conjugate | 3,000 | 1.96E−08 | | X | | X | | X | | X | | X | |
| Negative | 7 | C21 IT | 59 | 8.43E−10 | CTCL | X | X | X | X | X | X | X | X | X | X |
| control | | | | | | | | | | | | | | | |
| Conjugate | 7 | conjugate | 129 | 8.43E−10 | CTCL | X | X | X | X | X | X | X | X | X | X |
| matching | | | | | | | | | | | | | | | |

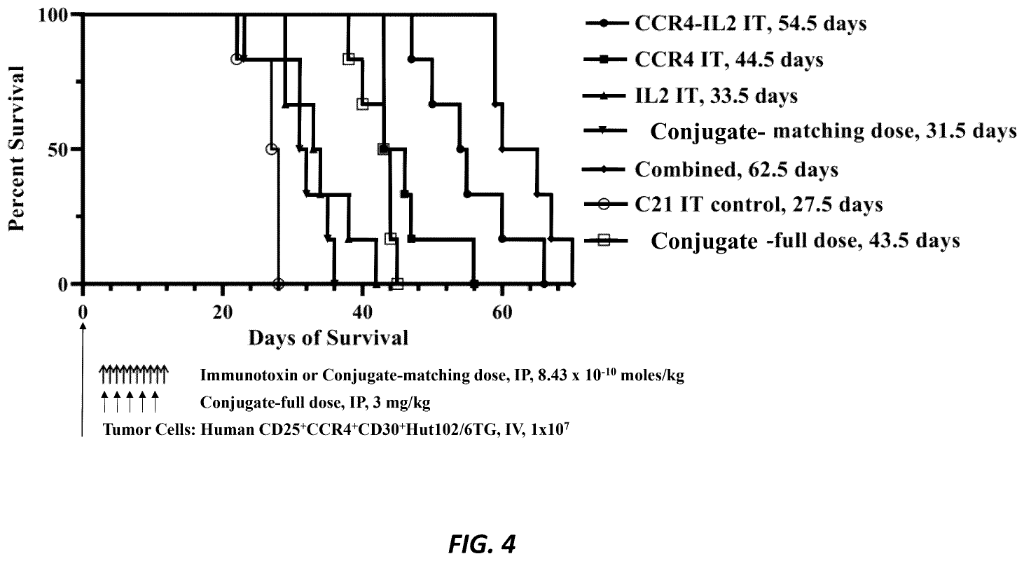

As shown in FIG. 4, Kaplan-Meier survival curves demonstrated that the survival of tumor-bearing animals in the CCR4-IL2 IT group was significantly longer than for animals of both conjugate full-dose and conjugate matching-dose groups. Animals of the combination treatment group (“combined”), treated with both CCR4-IL2 IT and the conjugate, survived longer than those of either the CCR4-1L2 IT or conjugate groups, alone, indicating a synergistic treatment effect achieved by administering both CCR4-IL2 IT and the conjugate. In addition, survival of animals in the conjugate full-dose group was comparable to that of animals in the CCR4 IT control group, indicating that the in vivo efficacy of a full dose of the conjugate is comparable with CCR4 IT.

The results from the frailty Cox proportional regression model were consistent with the above results. Compared to mice treated with conjugate full-dose, for instance, the hazards of death for mice treated with CCR4-IL2 IT and the CCR4-IL2 IT-conjugate combination were only 6.5% (95% CI, 2.2%, 19.5%) and 3% (95% CI, 0.9%-9.6%), respectively, of the hazard of those treated with conjugate full-dose. In addition, the hazard of death for mice treated with the combination was 45.5% of the hazard for mice treated with CCR4-IL2 IT with a marginal significance (p=0.058). These results indicate that both CCR4-IL2 IT and the CCR4-IL2 IT-conjugate combination prolong mice survival significantly compared to the conjugate, alone, and the combination approach is the most effective overall in a model of CTCL.

Pathology analysis was then conducted by surgically harvesting liver necropsy specimens on day 28 after animal euthanasia. In particular, a representative tumor-bearing animal from each treatment group was simultaneously euthanized for gross examination and pathology analysis. Tissues were fixed in 10% formalin, embedded in paraffin, and subsequently sectioned. Tissues were stained with hematoxylin and eosin for routine light microscopy. Slides were digitalized by an Aperio Scanscope (Leica, Wetzlar, Germany), and images were analyzed at 1.4× and 30× with Aperio ImageScope software (Leica).

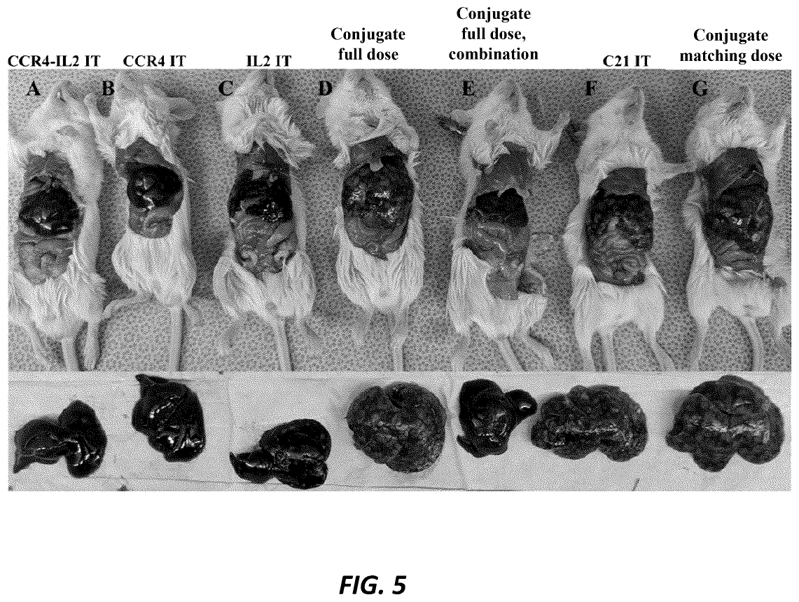

As shown in FIG. 5, liver sizes were normal for CCR4-IL2-, CCR4 IT-, and combination-treated animals. Zero white tumor nodules were observed on the liver surfaces of the combination-treated animals, and only a small number of white tumor nodules were observed on the surfaces of CCR4-IL2 IT- and CCR4 IT-treated animals. In contrast, visibly enlarged livers and extensive white tumor nodules were observed on the liver surfaces of the mice treated with C21 IT, the conjugate full-dose, and conjugate matching-dose. Tumor nodules were also observed on the liver surfaces of the 1L2 IT-treated animals. No difference in liver size or white tumor nodule number on the liver surface were observed between the conjugate full dose- and conjugate matching-dose-treated animals.

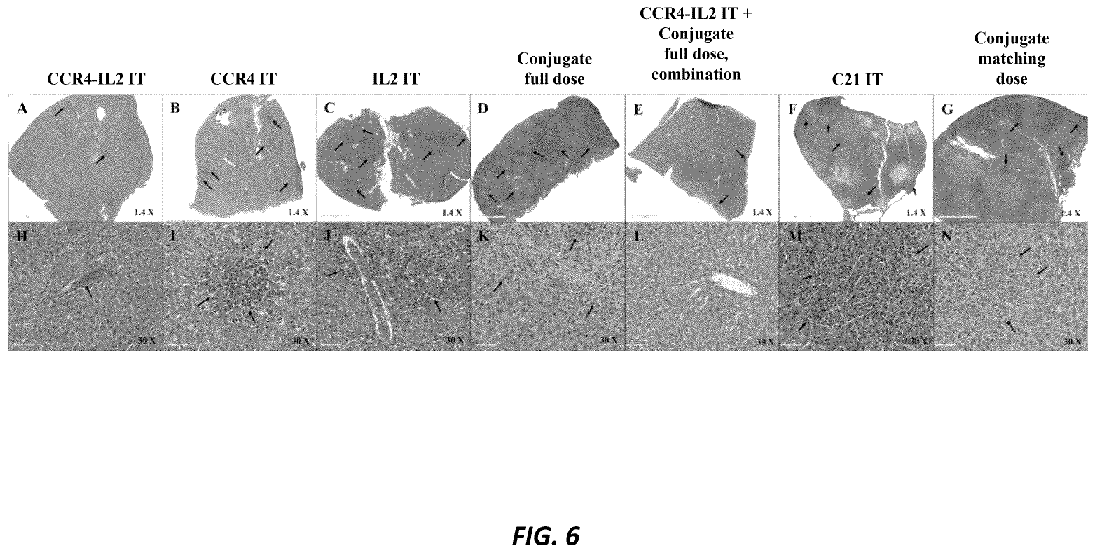

As shown in FIG. 6, additional pathological analysis demonstrated a complete lack of tumors in the examined liver sections of combination-treated animals (boxes E and L). Only a few small tumor cell nests were observed in the livers of CCR4-IL2 IT-treated animals (boxes A and H). In contrast, the tumor cell burden was mildly increased in CCR4 IT-treated animals (boxes B and I), and moderately increased in IL2 IT-treated animals (boxes C and J). The tumor burden was much higher in animals treated with the conjugate full-dose (boxes D and K), conjugate matching-dose (boxes G and N), and C21 IT (boxes F and M), with extensive replacement of normal liver tissues by each tumor. Gross examination and pathology data thus supported the Kaplan-Meier survival curves.

Accordingly, CCR4-IL2 IT was significantly more effective in prolonging the survival of mice having CTCL than the anti-CD30 antibody-drug conjugate, and a combination treatment involving the administration of both CCR4-IL2 IT and the anti-CD30 antibody-drug conjugate was more effective than either agent alone in an immunodeficient NSG mouse model of CTCL, indicating that administration of CCR4-IL2 IT alone or in combination with the anti-CD30 antibody-drug conjugate may effectively treat subjects afflicted with CTCL.

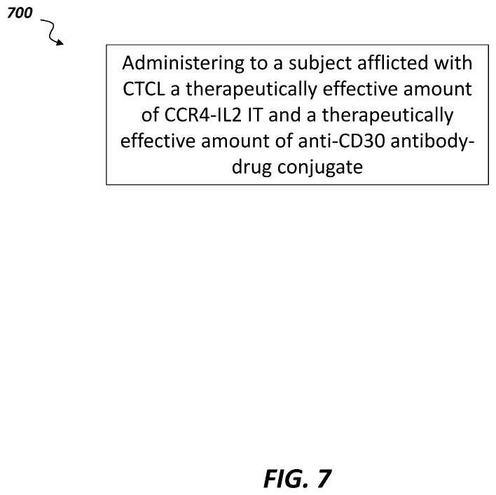

FIG. 7 illustrates an example method 700 of treating a subject afflicted with CTCL. As shown the non-limiting example method 700 involves administering to the subject a therapeutically effective amount of CCR4-IL2 IT and a therapeutically effective amount of anti-CD30 antibody-drug conjugate, which may comprise brentuximab. Together, CCR4-IL2 IT and the anti-CD30 antibody-drug conjugate may exhibit a surprising synergistic treatment effect.

While multiple embodiments are disclosed, still other embodiments of the present disclosure will become apparent to those skilled in the art from the foregoing description. As will be apparent, the disclosure is capable of modifications in various obvious aspects, all without departing from the spirit and scope of the present disclosure. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

What is claimed is:

1. A method of treating or alleviating at least one symptom of cutaneous T-cell lymphoma in a subject, comprising:
administering to the subject a therapeutically effective amount of a genetically engineered C-C motif chemokine receptor 4 bispecific immunotoxin (“CCR4-IL2 bispecific immunotoxin”) and a therapeutically effective amount of an anti-CD30 antibody-drug conjugate.

2. The method of claim 1, wherein the therapeutically effective amount of the CCR4-IL2 bispecific immunotoxin is administered via a first pharmaceutical composition.

3. The method of claim 2, wherein the therapeutically effective amount of the anti-CD30 antibody-drug conjugate is administered via a second pharmaceutical composition.

4. The method of claim 3, wherein the first pharmaceutical composition and the second pharmaceutical composition are administered concurrently.

5. The method of claim 3, wherein the first pharmaceutical composition and the second pharmaceutical composition are administered sequentially.

6. The method of claim 3, wherein the first pharmaceutical composition is administered at a higher dose than the second pharmaceutical composition.

7. The method of claim 1, wherein the genetically engineered CCR4-IL2 bispecific immunotoxin comprises an anti-human CCR4 scFv fused to a truncated diphtheria toxin DT390.

8. The method of claim 7, wherein the genetically engineered CCR4-IL2 bispecific immunotoxin comprises a human IL2 peptide domain.

9. The method of claim 1, wherein the therapeutically effective amount of the genetically engineered CCR4-IL2 bispecific immunotoxin and the therapeutically effective amount of the anti-CD30 antibody-drug conjugate is administered via a pharmaceutical composition.

10. The method of claim 1, wherein the cutaneous T-cell lymphoma comprises CCR4⁺ and CD25⁺ cutaneous T-cell lymphoma.

11. The method of claim 1, wherein the genetically engineered CCR4-IL2 bispecific immunotoxin reduces an amount of tumor-infiltrating Treg cells in the subject.

12. The method of claim 1, wherein the CCR4-IL2 bispecific immunotoxin and the anti-CD30 antibody-drug conjugate are administered intravenously.

13. The method of claim 1, further comprising performing a biopsy on a tumor within the subject.

14. The method of claim 1, wherein administering the CCR4-IL2 bispecific immunotoxin and the anti-CD30 antibody-drug conjugate causes a reduction in one or more of a tumor volume, tumor weight, tumor number, or tumor metastasis.

15. A system for treating or alleviating at least one symptom of cutaneous T-cell lymphoma in a subject diagnosed with cutaneous T-cell lymphoma, the system comprising:
at least one injection device configured to administer to the subject a therapeutically effective amount of a first pharmaceutical composition comprising a genetically engineered CCR4-IL2 bispecific immunotoxin and a therapeutically effective amount of a second pharmaceutical composition comprising an anti-CD30 antibody-drug conjugate.

16. The system of claim 15, wherein the genetically engineered CCR4-IL2 bispecific immunotoxin comprises an anti-human CCR4 scFv fused to a truncated diphtheria toxin DT390.

17. The system of claim 16, wherein the genetically engineered CCR4-IL2 bispecific immunotoxin comprises a human IL2 peptide domain.

18. The system of claim 15, wherein an amino acid sequence of the genetically engineered CCR4-IL2 bispecific immunotoxin is at least 90% identical to SEQ ID NO: 2.

19. The system of claim 15, wherein the first pharmaceutical composition is administered at a higher dose than the second pharmaceutical composition.

20. The system of claim 15, wherein the at least one injection device comprises an intravenous injection device.
